(19) United States
(12) Patent Application Publication
(10) Pub. No.: US 2010/0040559 A1
(43) Pub. Date: Feb. 18, 2010

(54) Use of a Pharmaceutical Composition Comprising an Anticholinergic for Killing Microorganisms and for Treating Respiratory Tract Infections

(75) Inventors: Stefan Leiner, Ockenheim (DE); Friedrich Schmidt, Ingelheim am Rhein (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim (DE)

(21) Appl. No.: 12/440,097

(22) PCT Filed: Aug. 22, 2007
(86) PCT No.: PCT/EP07/58722

§ 371 Date: Oct. 14, 2009

(30) Foreign Application Priority Data
Sep. 6, 2006 (EP) 06120196.8

(51) Int. Cl. A61K 9/12; A61K 31/5386

(52) U.S. Cl. 424/45; 514/230.5

(57) ABSTRACT

The present invention relates to the use of a compound of the formula 1 in which X⁻ is a singly negatively charged anion, R is H, methyl or OH, R¹ and R² are identical or different, phenyl or thienyl, for producing a medicament for killing microorganisms and/or for producing a medicament for the treatment of respiratory tract infections caused by microorganisms.

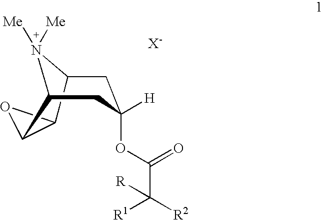

The present invention relates to the use of a compound of formula 1

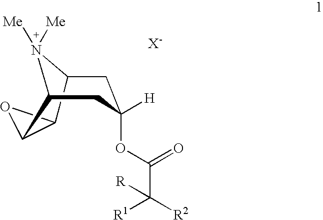

wherein

X⁻ denotes an anion with a single negative charge,

R denotes H, methyl or OH

R¹ and R² which may be identical or different represent phenyl or thienyl, for preparing a medicament for killing micro-organisms and/or for preparing a medicament for treating respiratory tract infections caused by micro-organisms.

The invention further relates to a pharmaceutical composition of a compound of the above formula 1, optionally in the form of the individual optical isomers, mixtures of the individual enantiomers or racemates, wherein the pharmaceutical composition does not contain any preservative.

The compounds of formula I are known from WO 02/32899 and from WO 91/04252. They have valuable pharmacological properties and as highly effective anticholinergics may provide a therapeutic benefit in the treatment of respiratory tract diseases, preferably in the treatment of inflammatory and/or obstructive respiratory tract diseases, particularly in the treatment of asthma or COPD (chronic obstructive pulmonary disease).

The aim of the present invention was to provide new uses for the compounds of formula 1.

Surprisingly, it has been found that the compounds of formula 1

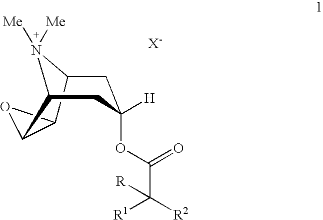

wherein
X⁻ denotes an anion with a single negative charge,
R denotes H, methyl or OH
R¹ and R² which may be identical or different represent phenyl or thienyl,

have a germicidal effect and can be used in particular for killing micro-organisms such as bacteria and fungi. It has also been found that the compounds of formula 1 are also suitable for preparing a medicament for the treatment of respiratory tract infections. A particular advantage according to the invention is the possibility of being able to treat both asthma or COPD and the respiratory tract infections that are frequently associated with these complaints using only a single active substance, namely a compound of formula 1.

The compounds of formula 1 are preferably administered by inhalation. Suitable inhalable powders may be used for this purpose, packed into suitable capsules (inhalettes) and administered using corresponding powder inhalers. Alternatively, they may be administered by inhalation using suitable inhalable aerosols. These also include powder inhalation aerosols which contain for example HFA134a, HFA227 or mixtures thereof as propellant gas, but also inhalable solutions, particularly aqueous, ethanolic or ethanolic/aqueous inhalable solutions.

US 2004/0209954 A1 describes how certain quaternary amines with an anticholinergic activity such as glycopyrrolate, mepenzolate and ipratropium have an antimicrobial activity, although none of the anticholinergics mentioned therein has a scopine structure. The scopine derivatives of formula 1, which are structurally very different from the anticholinergics glycopyrrolate, mepenzolate and ipratropium, are not mentioned in US 2004/0209954 A1, however.

Within the scope of the present invention, however, it is preferable to use those compounds of formula 1 wherein the anion X⁻ is selected from among the chloride, bromide, iodide, sulphate, phosphate, methanesulphonate, nitrate, maleate, acetate, citrate, fumarate, tartrate, oxalate, succinate, benzoate and p-toluenesulphonate.

Preferably, the salts of formula 1 are used wherein X⁻ denotes an anion selected from among the chloride, bromide, 4-toluenesulphonate and methanesulphonate.

Particularly preferred according to the present invention are those formulations which contain the compound of formula 1, wherein X⁻ denotes bromide.

References to the compound of formula 1 within the scope of the present invention also include all the possible amorphous and crystalline modifications of this compound. References to the compound of formula 1 within the scope of the present invention also include all the possible solvates and hydrates thereof which may be formed from this compound.

It is particularly preferred to use a compound according to formula 1, wherein R¹ and R² both denote phenyl, R denotes methyl and X⁻ denotes bromide (scopine 2,2-diphenylpropionate-methobromide), for killing micro-organisms and for preparing a medicament for the treatment of respiratory tract infections. This compound according to formula 1, wherein R¹ and R² both denote phenyl, R denotes methyl and X⁻ denotes bromide, is used to kill micro-organisms and to prepare a medicament for the treatment of respiratory tract infections, particularly preferably in aqueous solution for inhalation and in a concentration of 0.1 to 2.5 percent by weight, particularly from 0.2 to 1.5 percent by weight.

It is also particularly preferable to use a compound according to formula 1, wherein R¹ and R² both represent 2-thienyl, R denotes OH and X⁻ denotes bromide (tiotropium bromide), for killing micro-organisms and for preparing a medicament for the treatment of respiratory tract infections. This compound according to formula 1, wherein R¹ and R² both represent 2-thienyl, R denotes hydroxyl and X⁻ denotes bromide, is used to kill micro-organisms and to prepare a medicament for the treatment of respiratory tract infections, particularly preferably in aqueous solution for inhalation and in a concentration of 0.005 to 2.5 percent by weight, particularly from 0.01 to 0.5 percent by weight.

Preferably, the compounds according to formula 1 are used to kill micro-organisms selected from among the fungi and bacteria. The compounds according to formula 1 are more preferably used to kill bacteria selected from among Escherichia coli (e.g. ATCC 8739), Pseudomonas aeruginosa (e.g. ATCC 9027), Staphylococcus aureus (e.g. ATCC 6538), Bacillus subtilis (e.g. ATCC 6633), Streptococcus viridans (e.g. M1040), Streptococcus pyogenes (e.g. ATCC12344 or ATCC 19615), Neisseria spp., Streptococcus pneumoniae (e.g. ATCC 49 619), Corynebacterium spp., Pseudomonas spp. (e.g. Pseudomonas aeruginosa), Mycoplasma pneumoniae (e.g. ATCC 29342), Pseudomonas aeruginosa (e.g. ATCC 15442), Haemophilus influenzae (e.g. ATCC 51907 or ATCC 33391), Chlamydophila pneumoniae (e.g. ATCC VR 1310), Moraxella catarrhalis (e.g. ATCC 25238 or ATCC 8176), Enterobacteriaceae, Klebsiella pneumoniae (e.g. ATCC 10031 or ATCC 13883) and Bacillus spp. or to kill fungi selected from among Candida albicans (e.g. ATCC 10231) and Aspergillus niger (e.g. ATCC 16404). The compounds according to formula 1 are particularly preferably used to kill bacteria selected from among Escherichia coli (e.g. ATCC 8739), Staphylococcus aureus (e.g. ATCC 6538), Bacillus subtilis (e.g. ATCC 6633) and to kill fungi selected from among Candida albicans (e.g. ATCC 10231) and Aspergillus niger (e.g. ATCC 16404).

Another preferred embodiment of the present invention relates to the use of the compounds of formula 1 for preparing a medicament for treating respiratory tract infections. These respiratory tract infections are preferably caused by bacteria selected from among Escherichia coli (e.g. ATCC 8739), Pseudomonas aeruginosa (e.g. ATCC 9027), Staphylococcus aureus (e.g. ATCC 6538), Bacillus subtilis (e.g. ATCC 6633), Streptococcus viridans (e.g. M1040), Streptococcus pyogenes (e.g. ATCC12344 or ATCC 19615), Neisseria Spp., Streptococcus pneumoniae (e.g. ATCC 49 619), Corynebacterium spp., Pseudomonas spp. (e.g. Pseudomonas aeruginosa), Mycoplasma pneumoniae (e.g. ATCC 29342), Pseudomonas aeruginosa (e.g. ATCC 15442), Haemophilus influenzae (e.g. ATCC 51907 or ATCC 33391), Chlamydophila pneumoniae (e.g. ATCC VR 1310), Moraxella catarrhalis (e.g. ATCC 25238 or ATCC 8176), Enterobacteriaceae, Klebsiella pneumoniae (e.g. ATCC 10031 or ATCC 13883) and Bacillus spp, but particularly by bacteria selected from among Escherichia coli (e.g. ATCC 8739), Pseudomonas aeruginosa (e.g. ATCC 9027), Staphylococcus aureus (e.g. ATCC 6538), Bacillus subtilis (e.g. ATCC 6633). These respiratory tract infections are also preferably caused by fungi, particularly selected from among Candida albicans (e.g. ATCC 10231) and Aspergillus niger (e.g. ATCC 16404). It is particularly preferable to use the compounds of formula 1 to prepare a medicament for treating respiratory tract infections caused by the fungi Aspergillus niger and Candida albicans or by a bacterium selected from among Pseudomonas aeruginosa or Staphylococcus aureus, Bacillus subtilis and Escherichia coli.

In another preferred embodiment the invention relates to a pharmaceutical composition containing a compound of formula 1

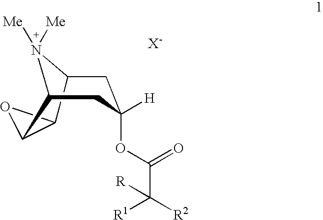

wherein

X⁻ denotes an anion with a single negative charge,

R denotes H, methyl or OH

R¹ and R² which may be identical or different represent phenyl or thienyl, optionally in the form of the individual optical isomers, mixtures of the individual enantiomers or racemates, wherein the pharmaceutical composition does not contain any additional preservative.

Within the scope of the present invention, preferably those compounds of formula 1 are used wherein the anion X⁻ is selected from among the chloride, bromide, iodide, sulphate, phosphate, methanesulphonate, nitrate, maleate, acetate, citrate, fumarate, tartrate, oxalate, succinate, benzoate and p-toluenesulphonate.

Preferably, the salts of formula 1 are used wherein X⁻ denotes an anion selected from among the chloride, bromide, 4-toluenesulphonate and methanesulphonate.

Particularly preferred within the scope of the present invention are those formulations that contain the compound of formula 1, wherein X⁻ denotes bromide.

The compounds of formula 1 according to the invention are preferably administered by inhalation. Suitable inhalable powders packed into suitable capsules (inhalettes) may be administered using suitable powder inhalers. Alternatively, the drug may be inhaled by the application of suitable inhalation aerosols. These also include powdered inhalation aerosols which contain for example HFA134a, HFA227 or a mixture thereof as propellant gas, and naturally also propellant-containing or propellant-free inhalable solutions of the compounds according to formula 1 in a solvent selected from among water, ethanol and a water/ethanol mixture, while aqueous inhalable solutions are preferred and aqueous propellant-free inhalable solutions are particularly preferred.

In a particularly preferred embodiment, the invention relates to a pharmaceutical composition containing a compound according to formula 1, wherein R¹ and R² both represent phenyl, R denotes methyl and X⁻ denotes bromide (scopine 2,2-diphenylpropionate methobromide), optionally in the form of the individual optical isomers, mixtures of the individual enantiomers or racemates, characterised in that the pharmaceutical composition does not contain any preservative. This pharmaceutical composition is preferably an aqueous, more particularly a propellant-free aqueous aerosol formulation containing a scopine 2,2-diphenylpropionate methobromide.

The scopine 2,2-diphenylpropionate methobromide is preferably present in the aqueous formulation in a concentration of 0.1 to 2.5 percent by weight, more particularly in a concentration of 0.2 to 1.5 percent by weight. The pH of this preferred formulation is preferably, according to the invention, within the range from 2.5 and 6.5, preferably in the range from 3.0 to 5.0, more preferably in the range from 3.5 to 4.5, especially in the range from 3.6 to 4.4.

In another particularly preferred embodiment the invention relates to a pharmaceutical composition containing a compound according to formula 1, wherein R¹ and R² denote 2-thienyl, R denotes hydroxy and X⁻ denotes bromide (tiotropium bromide), optionally in the form of the individual optical isomers, mixtures of the individual enantiomers or racemates, characterised in that the pharmaceutical composition does not contain any preservative. This pharmaceutical composition is preferably an aqueous, more particularly a propellant-free aerosol formulation containing tiotropium bromide.

Tiotropium bromide is preferably present in the aqueous aerosol formulation in a concentration of 0.005 to 2.5 percent by weight, particularly in a concentration of 0.01 to 0.5 percent by weight. The pH of the formulation according to the invention is between 2.0 and

4.5, preferably between 2.5 and 3.5 and more preferably between 2.7 and 3.3 and still more preferably between 2.7 and 3.2. Most preferably, the pH has an upper limit of 3.1.

The preferred aqueous propellant-free aerosol formulations preferably additionally contain the acid(s) needed to adjust the pH.

The pH is adjusted by the addition of pharmacologically acceptable acids. Examples of preferred inorganic acids for this purpose are: hydrochloric acid, hydrobromic acid, nitric acid, sulphuric acid and/or phosphoric acid. Examples of particularly suitable organic acids are: ascorbic acid, citric acid, malic acid, tartaric acid, maleic acid, succinic acid, fumaric acid, acetic acid, formic acid and/or propionic acid, etc. Preferred inorganic acids are hydrochloric acid and sulphuric acid. It is also possible to use acids which form an acid addition salt with the active substance. Of the organic acids, ascorbic acid, fumaric acid and citric acid are preferred, especially citric acid. If desired, mixtures of the abovementioned acids may also be used, particularly in the case of acids which have other properties in addition to their acidifying properties, e.g. those which act as flavourings or antioxidants, such as for example citric acid or ascorbic acid. Hydrochloric acid deserves special mention as an inorganic acid.

If desired, pharmacologically acceptable bases may also be used to titrate the pH precisely. Suitable bases include for example alkali metal hydroxides and alkali metal carbonates. The preferred alkali ion is sodium. If bases of this kind are used, care must be taken to ensure that the resulting salts, which are then contained in the finished pharmaceutical formulation, are pharmacologically compatible with the abovementioned acid.

The preferred aqueous, propellant-free aerosol formulations may additionally contain, besides water as the solvent and the compound of formula 1, a complexing agent, particularly sodium edetate, other cosolvents and other adjuvants and additives such as e.g. antioxidants, surfactants, etc., with the exception of preservatives. In another embodiment of the aqueous propellant-free aerosol formulations, no additives at all are present apart from the compound according to formula 1 and water as the solvent.

By complexing agents are preferably meant within the scope of the present invention molecules which are capable of entering into complex bonds. Preferably, these compounds should have the effect of complexing cations, most preferably metal cations.

The formulations according to the invention preferably contain editic acid (EDTA) or one of the known salts thereof, e.g. sodium EDTA or disodium EDTA, as complexing agent. Preferably, sodium edetate and disodium edetate are used, optionally in the form of the hydrates, more preferably in the form of the dihydrates thereof. If complexing agents are used within the formulations according to the invention, their content is preferably in the range from 5 to 20 mg per 100 ml, more preferably in the range from 7 to 15 mg per 100 ml of the formulation according to the invention. Particularly preferably, the formulations according to the invention contain a complexing agent, preferably sodium edetate or disodium edetate or one of the hydrates thereof, in an amount of about 9 to 12 mg per 100 ml, more preferably about 10 mg per 100 ml of the formulation according to the invention.

The remarks made concerning sodium edetate and disodium edetate also apply analogously to other possible additives which are comparable to EDTA or the salts thereof, which have complexing properties and can be used instead of them, such as for example nitrilotriacetic acid and the salts thereof.

Other pharmacologically acceptable adjuvants may also be added to the formulation according to the invention. By adjuvants and additives are meant, in this context, any pharmacologically acceptable and therapeutically useful substance which is not an active substance, but can be formulated together with the active substance in the pharmacologically suitable solvent, in order to improve the qualities of the active substance formulation. Preferably, these substances have no pharmacological effects or no appreciable or at least no undesirable pharmacological effects in the context of the desired therapy. The adjuvants and additives include, for example, stabilisers, antioxidants and/or preservatives which prolong the shelf life of the finished pharmaceutical formulation, as well as flavourings, vitamins and/or other additives known in the art. The additives also include pharmacologically acceptable salts such as sodium chloride, for example.

The preferred excipients include antioxidants such as ascorbic acid, for example, provided that it has not already been used to adjust the pH, vitamin A, vitamin E, tocopherols and similar vitamins or provitamins occurring in the human body.

Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters, provided that they are not the solvent or suspending agent.

The terms adjuvants and additives in this context denote any pharmacologically acceptable and therapeutically useful substance which on the one hand is not an active substance and on the other hand is not a preservative, but which can be formulated with the active substance in the pharmacologically suitable solvent in order to improve the qualitative properties of the active substance formulation. Preferably, these substances have no pharmacological effect or, in connection with the desired therapy, no appreciable or at least no undesirable pharmacological effect. The adjuvants and additives include, for example, surfactants such as soya lecithin, oleic acid, sorbitan esters, such as sorbitan trioleate, polyvinylpyrrolidone, other stabilisers, complexing agents, antioxidants, flavourings, vitamins and/or other additives known in the art. The additives also include pharmacologically acceptable salts such as sodium chloride.

The germicidal effect of the compounds according to formula 1 can be demonstrated by comparing test solutions (=aqueous solutions which contain the compound according to formula 1, with placebo solutions (=identical aqueous solutions without the compound according to formula 1) using the test method as described in European Pharmacopoeia 4 (2002), General Text 5.1.3, Efficacy of Antimicrobial Preservation. Defined volumes of the test solutions and the placebo solutions are each combined with identical amounts of an inoculating solution of a particular micro-organism, homogenised and incubated at a temperature and humidity that are suitable for the growth of the micro-organism. At defined intervals (e.g. after 1 day, 2 days, 3 days, 7 days, 14 days and 28 days) the number of terminative organisms (“colony forming units”) in the inoculated formulations is measured and the results obtained for the test solution and placebo solution are compared.

To determine the number of germinative organisms (“colony forming units”) in the inoculated formulations samples are taken from these formulations at specified intervals and diluted with TSB medium (Medium A) containing Inactivator No. 5 (3% Tween 80, 0.3% lecithin, 0.1% histidine). Then these dilutions are subjected to membrane filtration. To determine the number of bacteria or fungi, the membrane filters were transferred successively onto TSA medium (Medium B) and Sabourraud agar (Medium C). The TSA plates were incubated for 3-5 days at 30-35° C. and the Sabourraud agar plates at 20-25° C. for five days in each case. Then the colonies are counted on the two plates.

1. A method for killing microorganisms comprising administering to a patient in need thereof a pharmaceutically acceptable amount of a compound of formula 1 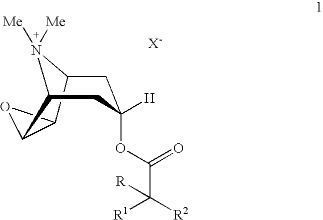
wherein
X⁻ denotes an anion with a single negative charge,
R denotes H, methyl or OH
R¹ and R² which may be identical or different represent phenyl or thienyl,
optionally in the form of the individual optical isomers, mixtures of the individual enantiomers or racemates.

2. A method for treating respiratory tract infections caused by microorganisms comprising administering to a patient in need thereof a pharmaceutically acceptable amount of a compound of formula 1 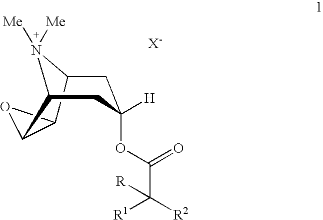
wherein
X⁻ denotes an anion with a single negative charge,
R denotes H, methyl or OH
R¹ and R² which may be identical or different represent phenyl or thienyl,
optionally in the form of the individual optical isomers, mixtures of the individual enantiomers or racemates.

3. The method according to claim 1 or 2, wherein the compound of formula 1 is administered by inhalation.

4. The method according to claim 1 or 2, wherein R¹ and R² both represent phenyl, R denotes methyl and X⁻ denotes bromide.

5. The method according to claim 4, wherein the compound according to formula 1 is present in an aqueous solution in a concentration of 0.1 to 2.5 percent by weight.

6. The method according to claim 5, wherein the compound according to formula 1 is present in an aqueous solution in a concentration of 0.2 to 1.5 percent by weight.

7. The method according to claim 1 or 2, wherein R¹ and R² denote 2-thienyl and R denotes OH.

8. The method according to claim 7, wherein the compound according to formula 1 is present in a concentration of 0.005 to 2.5 percent by weight.

9. The method according to claim 8, wherein the compound according to formula 1 is present in a concentration of 0.01 to 0.5 percent by weight.

10. The method according to claim 1 or 2, wherein the microorganisms are fungi or bacteria.

11. The method according to claim 10, wherein the microorganisms are bacteria selected from the group consisting of: Escherichia coli, Pseudomonas aeruginosa, Staphylococcus aureus, Bacillus subtilis, Streptococcus viridans, Streptococcus pyogenes, Neisseria spp., Streptococcus pneumoniae, Corynebacterium spp., Pseudomonas spp., Mycoplasma pneumoniae, Pseudomonas aeruginosa, Haemophilus influenzae, Chlamydophila pneumoniae, Moraxella catarrhalis, Enterobacteriaceae, Klebsiella pneumoniae and Bacillus spp.

12. The method according to claim 10, wherein the microorganisms are fungi selected from the group consisting of: Candida albicans and Aspergillus niger.

13. A pharmaceutical composition containing a compound of formula 1 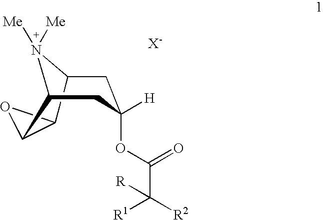
wherein
X⁻ denotes an anion with a single negative charge,
R denotes H, methyl or OH
R¹ and R² which may be identical or different represent phenyl or thienyl, optionally in the form of the individual optical isomers, mixtures of the individual enantiomers or racemates, wherein the pharmaceutical composition does not contain any additional preservative.

14. The pharmaceutical composition according to claim 13, wherein the pharmaceutical composition is an aqueous aerosol formulation.

15. The aqueous aerosol formulation according to claim 14, wherein R¹ and R² both represent phenyl, R denotes methyl and X⁻ denotes bromide and the aqueous aerosol formulation is administered via inhalation.

16. The aqueous aerosol formulation according to claim 15, wherein the compound according to formula 1 is present in a concentration of 0.1 to 2.5 percent by weight.

17. The aqueous aerosol formulation according to claim 16, wherein the compound according to formula 1 is present in a concentration of 0.2 to 1.5 percent by weight.

18. The aqueous aerosol formulation according to claim 14, wherein R¹ and R² both represent 2-thienyl, R denotes OH and X⁻ denotes bromide and the aqueous aerosol formulation is administered via inhalation.

19. The aqueous aerosol formulation according to claim 18, wherein the compound according to formula 1 is present in a concentration of 0.005 to 2.5 percent by weight.

20. The aqueous aerosol formulation according to claim 19, wherein the compound according to formula 1 is present in a concentration of 0.1 to 0.5 percent by weight.
